SEQUENCE LISTING (29 sequences)

SEQ ID NO: 1 - DNA, 20 bp, synthetic construct
  source 1..20: mol_type = other DNA; note = PDSsgRNA1
ggaacaacgagatgctgaca

SEQ ID NO: 2 - DNA, 20 bp, synthetic construct
  source 1..20: mol_type = other DNA; note = PDSsgRNA2
gctgcatggaaggatgaaga

SEQ ID NO: 3 - DNA, 22 bp, synthetic construct
  source 1..22: mol_type = other DNA; note = Primer Bra032770F
agaacaaggcgaaggactgctg

SEQ ID NO: 4 - DNA, 22 bp, synthetic construct
  source 1..22: mol_type = other DNA; note = Primer Bra032770R
tgcattgacagttcgttcgggt

SEQ ID NO: 5 - DNA, 22 bp, synthetic construct
  source 1..22: mol_type = other DNA; note = Primer Bra010751F
aacaaggaggaggaggagtgct

SEQ ID NO: 6 - DNA, 22 bp, synthetic construct
  source 1..22: mol_type = other DNA; note = Primer Bra010751R
tgtcacccgatcaggtactccc

SEQ ID NO: 7 - DNA, 4332 bp, synthetic construct
  source 1..4332: mol_type = other DNA; note = 35S-ZmWUS2-P2A-AtIPT-P2A-AtPLT5-NOS expression framework
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgggaagcggagctactaacttcagcctgctgaagcaggctggagacgtggaggagaaccctggacctatggatctgcgtctaattttcggtccaacttgcacaggaaagacgtcgaccgcgatacgtcttgcccagcagactggccttccagtcctttcgctcgatcgggtccaatgctgtcctcaactgtcaaccggaagcggacgaccaacagtggaagaactgaaaggaacgacccgtctataccttgaagatcggcctctggtgaagggtatcatcgcagccaagcaagctcacgaaaggctgatcggggaagtgtacaattatgaggcccacggcgggcttattcttgagggaggatctatctcgttgctcaggtgcatggcgcaaagcagttattggagtaccgattttcgttggcatattattcgccacaagttagcagacgaggagacattcatgaacgcggccaaggccagagttaggcagatgttgcgccctgctgtaggcccatctattattcaagagttggttcatctttggaatgagcctcggctgaggcccatactgaaagagatcgacggatatcgatatgccatgttatttgctagccagaaccagatcacacccgatatgctattgcagcttgacccagatatggagggtgagttgattcatggaatcgctcaggagtatctcatccatgcgcgccggcaggagcaggagttccctccagtgagcgtggtcgctttcgaaggattcgaaggtccaccgttcggaatgtgcggaagcggagctactaacttcagcctgctgaagcaggctggagacgtggaggagaaccctggacctatgaagaacaataacaacaaatcttcttcttcttctagctatgattcttctttgtctccttcttcttcatcctcctcccaccagaactggctctctttctctctctccaacaataacaacaacttcaattcttcctcaaaccctaatctcacttcctccacatcagatcatcatcatcctcacccttctcacctctctctctttcaagctttctccacttctccagtcgaacggcaagatgggtcaccgggagtttcacccagcgatgccacggcggttctttccgtataccccggcggtcctaaacttgagaacttcctcggcggaggagcctcaacgacgacaacaagaccaatgcaacaagtgcaatctcttggcggcgttgtcttctcttccgacctacagccaccgcttcatcctccgtccgccgccgagatctacgactctgagctcaagtcaatagccgctagcttcctaggaaactactccggtggacactcgtcggaggtctctagcgtacataaacaacaaccgaatcctctagctgtctcagaggcttcgcctactccgaagaagaacgtagagagttttggacaacgtacctcgatttatagaggagtcacaagacatagatggactggaagatacgaagctcatctatgggataatagttgccgaagagaaggccaaagcagaaaaggaagacaagtttatttaggtggttatgataaggaagataaagcagctagagcttacgaccttgcagctcttaagtattggggtcctacaactacgactaatttcccgatatcaaattacgaatctgaacttgaagaaatgaaacacatgactcgacaagagttcgttgcttctttaagacggaaaagcagtggattctctaggggtgcctccatgtacagaggcgtcactagacatcatcagcatggtcgatggcaggcacgaattggaagagttgcaggcaacaaagacctttatcttggcacatttagcactcaagaggaagctgcagaagcttatgatatagcagcgatcaaattccgcggtctaaatgcagtcaccaatttcgacatcagtcgatatgatgtcaaatcaattgctagctgtaatctccctgtgggtggactaatgcctaaaccttctccagcaaccgcagcggctgacaaaaccgttgatctttctccatccgactctccatctctaaccacaccgtccctcacgttcaatgtggcaacaccggtcaatgaccatggaggaactttttaccacactggtataccaatcaaaccagacccggctgatcattattggtccaacatctttggattccaggcaaacccgaaagcagaaatgcgaccattagcaaactttgggtcggatcttcataacccttctcctggttatgctataatgccggtaatgcaggaaggtgaaaacaactttggtggtagttttgttgggtctgatgggtataacaatcattccgctgcatcgaacccggtctcagcaattccgctgtcctcgacaactacaatgagtaacggtaacgaagggtatggtggaaacataaactggattaataacaacatttcaagttcttaccaaactgcaaaatcaaatctctctgttttgcacacaccggtttttgggttggaatagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 8 - DNA, 23 bp, synthetic construct
  source 1..23: mol_type = other DNA; note = Fragment of WT-PDS1
gctgcatggaaggatgaagatgg

SEQ ID NO: 9 - DNA, 40 bp, synthetic construct
  source 1..40: mol_type = other DNA; note = Fragment of WT-PDS1
ggaacaacgagatgctgacatggccagagaaaataaagtt

SEQ ID NO: 10 - DNA, 23 bp, synthetic construct
  source 1..23: mol_type = other DNA; note = Fragment of WT-PDS2
gctgcatggaaggatgaagatgg

SEQ ID NO: 11 - DNA, 40 bp, synthetic construct; identical to SEQ ID NO: 9
  source 1..40: mol_type = other DNA; note = Fragment of WT-PDS2

SEQ ID NO: 12 - DNA, 24 bp, synthetic construct
  source 1..24: mol_type = other DNA; note = Fragment of A-PDS1
gctgcatggaaggatgtgacatgg

SEQ ID NO: 13 - DNA, 17 bp, synthetic construct
  source 1..17: mol_type = other DNA; note = Fragment of A-PDS1
ccagagaaaataaagtt

SEQ ID NO: 14 - DNA, 20 bp, synthetic construct
  source 1..20: mol_type = other DNA; note = Fragment of A-PDS2
gctgcatggaaggaagatgg

SEQ ID NO: 15 - DNA, 40 bp, synthetic construct
  source 1..40: mol_type = other DNA; note = Fragment of A-PDS2
ggaacaacgagatgctgaacatggccagagaaaataaagt

SEQ ID NO: 16 - DNA, 20 bp, synthetic construct
  source 1..20: mol_type = other DNA; note = Fragment of B-PDS1
gctgcatggaaggaagatgg

SEQ ID NO: 17 - DNA, 40 bp, synthetic construct; identical to SEQ ID NO: 9
  source 1..40: mol_type = other DNA; note = Fragment of B-PDS1

SEQ ID NO: 18 - DNA, 20 bp, synthetic construct
  source 1..20: mol_type = other DNA; note = Fragment of B-PDS2
gctgcatggaaggaagatgg

SEQ ID NO: 19 - DNA, 40 bp, synthetic construct; identical to SEQ ID NO: 9
  source 1..40: mol_type = other DNA; note = Fragment of B-PDS2

SEQ ID NO: 20 - DNA, 20 bp, synthetic construct
  source 1..20: mol_type = other DNA; note = Fragment of C-PDS1
gctgcatggaaggaagatgg

SEQ ID NO: 21 - DNA, 40 bp, synthetic construct; identical to SEQ ID NO: 9
  source 1..40: mol_type = other DNA; note = Fragment of C-PDS1

SEQ ID NO: 22 - DNA, 20 bp, synthetic construct
  source 1..20: mol_type = other DNA; note = Fragment of C-PDS2
gctgcatggaaggaagatgg

SEQ ID NO: 23 - DNA, 40 bp, synthetic construct; identical to SEQ ID NO: 9
  source 1..40: mol_type = other DNA; note = Fragment of C-PDS2

SEQ ID NO: 24 - DNA, 20 bp, synthetic construct
  source 1..20: mol_type = other DNA; note = Fragment of D-PDS1
gctgcatggaaggaagatgg

SEQ ID NO: 25 - DNA, 40 bp, synthetic construct
  source 1..40: mol_type = other DNA; note = Fragment of D-PDS1
gaaacaacgagatgctgaacatggccagagaaaataaagt

SEQ ID NO: 26 - DNA, 20 bp, synthetic construct
  source 1..20: mol_type = other DNA; note = Fragment of D-PDS2
gctgcatggaaggaagatgg

SEQ ID NO: 27 - DNA, 10 bp, synthetic construct
  source 1..10: mol_type = other DNA; note = Fragment of D-PDS2
gtcggaggtc

SEQ ID NO: 28 - DNA, 20 bp, synthetic construct
  source 1..20: mol_type = other DNA; note = Fragment of E-PDS2
gctgcatggaaggaagatgg

SEQ ID NO: 29 - DNA, 40 bp, synthetic construct; identical to SEQ ID NO: 9
  source 1..40: mol_type = other DNA; note = Fragment of E-PDS2

(19) United States
(12) Patent Application Publication
(10) Pub. No.: US 2026/0078389 A1
(43) Pub. Date: Mar. 19, 2026

(54) METHOD FOR EFFICIENT GENETIC TRANSFORMATION AND GENE EDITING OF BRASSICA CROP MEDIATED BY AGROBACTERIUM RHIZOGENES

(71) Applicants: NANJING AGRICULTURAL UNIVERSITY, Nanjing, Jiangsu (CN); NJ Tianliang Bioengineering Technology Co., Ltd., Jiangning District Nanjing, Jiangsu (CN); Nanjing Suman Plasma ERI. Co., Ltd., Jiangning District Nanjing, Jiangsu (CN)

(72) Inventors: Xilin HOU, Nanjing, Jiangsu (CN); Changwei ZHANG, Nanjing, Jiangsu (CN); Yaolong WANG, Nanjing, Jiangsu (CN); Tongkun LIU, Nanjing, Jiangsu (CN); Dong XIAO, Nanjing, Jiangsu (CN); Ying LI, Nanjing, Jiangsu (CN); Jianjun WANG, Nanjing, Jiangsu (CN); Chunmei HU, Nanjing, Jiangsu (CN)

(21) Appl. No.: 18/877,624

(22) PCT Filed: Nov. 4, 2024
(86) PCT No.: PCT/CN2024/129620

§ 371 Date: Dec. 20, 2024

(30) Foreign Application Priority Data
May 8, 2024 (CN) 202410562568.6

(51) Int. Cl. C12N 15/82; C07K 14/415; C12N 9/22; C12N 15/11

(52) U.S. Cl. CPC C12N 15/8205; C07K 14/415; C12N 9/226; C12N 15/111; C12N 15/8212; C12N 2310/20

(57) ABSTRACT

A method for efficient genetic transformation and gene editing of a Brassica crop mediated by Agrobacterium rhizogenes is provided. Genetic transformation of the Brassica crop and establishment of a gene editing system are achieved by Agrobacterium rhizogenes-mediated delivery of three developmental regulators (DRs), ZmWUS2, AtIPT, and AtPLT5. The Agrobacterium rhizogenes-mediated delivery of three DRs, ZmWUS2, AtIPT, and AtPLT5, may induce explants to form callus and then directly form buds. The method solves the problem of difficult root regeneration for the Brassica crop, and enables species with a poor root regeneration ability to achieve efficient transformation.

### CROSS-REFERENCE TO RELATED APPLICATION

This patent application is the US national phase entry of international patent application PCT/CN2024/129620, filed on Nov. 4, 2024, which claims the benefit and priority of Chinese Patent Application No. 202410562568.6 filed with the China National Intellectual Property Administration on May 8, 2024, the disclosure of which is incorporated by reference herein in its entirety as part of the present application.

### REFERENCE TO SEQUENCE LISTING

A computer readable XML filed entitled GWPCTP20240806331 seqlist″″, that was created on Nov. 28, 2024, with a file size of about 36,219 bytes, contains the sequence listing for this application, has been filed with this application, and is hereby incorporated by reference in its entirety.

### TECHNICAL FIELD

The present disclosure belongs to the technical field of plant genetic engineering, and particularly relates to a method for efficient genetic transformation and gene editing of a Brassica crop mediated by Agrobacterium rhizogenes.

### BACKGROUND

In the past period of time, many achievements have been made in the gene function verification, disease resistance, and stress resistance of Brassica crops. However, existing functional studies are mostly based on transient expression, gene gun bombardment, and Agrobacterium tumefaciens-mediated genetic transformation, all of which have considerable limitations. In fact, most elite Brassica crops are extremely inefficient against infection by Agrobacterium tumefaciens. Moreover, obtaining transgenic seedlings through tissue culture is highly dependent on genotype, and complex hormone combinations make the tissue culture more challenging. With the rapid advance of genome sequencing technology and the birth of gene editing technology, genetic transformation has become an important biotechnology for studying gene function and breeding. Therefore, it is of great significance to develop an efficient and stable genetic transformation system for Brassica crops.

Decades ago, some Brassica crop subspecies could be genetically transformed, but the low transformation efficiency prevented the genetic transformation system from being applied to gene function research. Currently, researchers have not established an efficient genetic transformation system for Brassica crops. With the development of clustered regularly interspaced short palindromic repeats (CRISPR), the existing genetic transformation system of Brassica crops cannot meet the requirements well, and seriously affects the progress of plant breeding and gene editing. In recent years, with the introduction of the cut-dip-budding (CDB) delivery system, Agrobacterium rhizogenes-mediated infection to organs such as hypocotyls, stem segments, and aerial parts of the plants without roots has been ultized, employing different plant hormone combinations to induce transgenic roots, thereby obtaining regenerated transgenic plants. However, for Brassica crops, although infection with Agrobacterium rhizogenes can produce stable composite plants (with transgenic roots and non-transgenic aerial parts), it is extremely challenging to induce bud regeneration using the transgenic roots of Brassica crops.

Plant cells exhibit strong plasticity and omnipotence, which makes it possible to obtain new plants through tissue culture. Tissue culture regeneration is a process that involves inducing callus from various explants such as leaves, petioles, hypocotyls, roots, and microspores, inducing bud regeneration with the callus, and finally rooting to obtain a complete plant. Over the past many years, increasing evidence has shown that the totipotency of plant somatic cells for in vitro regeneration is regulated by a complex network. The fate of plant cells can be determined and switched by a series of developmental regulators (DRs). For example, researchers have completed genetic transformation of genetically-stubborn wheat using TaWOX5 gene: the chimeric fusion of CIGRF 4 and CIGIF 1 has achieved efficient genetic transformation of watermelon; DRs such as WUS, WIND1, and PLT 5 have promoted plant genetic transformation of snapdragon; PLT5 and WUS have been shown to promote plant genetic transformation of tomatoes; and PLT5 can also promote the transformation of Brassica rapa ssp. chinensis. Research results show that plant developmental regulatory genes play a key role in callus formation and plant regeneration. The most famous developmental regulatory gene is WUSCHEL (WUS), which is extremely important in promoting the organogenesis and embryogenesis. It is also found that auxin biosynthesis genes YUCCA1 (YUCI) and YUC4 are activated through PLTs (PLT 3, PLT 5, and PLT 7), resulting in the expression of a shoot-promoting factor CUP-SHAPED COTYLEDON2 (CUC2) and then leading to bud regeneration.

Recently, researchers have obtained transgenic roots of fruit trees through Agrobacterium rhizogenes, followed by using the transgenic roots to induce buds to obtain transgenic plants. The CDB delivery system established by Cao et al. allows for direct obtainment of transgenic plants through in vitro infection, and is highly effective for species with strong root regeneration ability. However, Brassica crops exhibit almost no root regeneration ability.

### SUMMARY

A first objective of the present disclosure is to provide a gene editing vector, including a clustered regularly interspaced short palindromic repeats-associated protein 9 (Cas9) vector backbone and DRs, the DRs being ZmWUS2, AtIPT, and AtPLT5.

A second objective of the present disclosure is to provide a gene editing system, where a single guide RNA (sgRNA) of a target gene is ligated to the gene editing vector.

A third objective of the present disclosure is to provide use of the gene editing vector or the gene editing system in genetic transformation, gene editing, and/or tissue culture of a Brassica crop.

A fourth objective of the present disclosure is to provide a method for genetic transformation and/or gene editing of a Brassica crop, including: ligating an sgRNA designed based on a sequence from a target gene of the Brassica crop with the gene editing vector, transforming a resulting ligation product into Agrobacterium rhizogenes to infect an explant of the Brassica crop, and then subjecting the explant to callus induction and adventitious bud regeneration in sequence to obtain a gene-edited plant.

### BRIEF DESCRIPTION OF THE DRAWINGS

FIGS. 1A-1B shows structural schematic diagrams of the gene editing vector and the gene editing system in the present disclosure;

FIG. 2 shows a process of callus and bud regeneration in Example 1: where (a) shows seed culture: (b) shows explants: (c) shows Agrobacterium rhizogenes infection: (d) shows callus induction, and red callus is positive: (e) shows that the red callus is cut and subcultured to induce adventitious buds: (f) shows that the adventitious buds develop into plants and induce rooting: (g) shows that positive plants are marked with red (left), and green plants are wild type (right); and (h) shows that transgenic plants are transplanted; and

FIGS. 3A-3B show effect of PDS gene and editing site sequence alignment of the pakchoi (Brassica rapa ssp. chinensis) in Example 1: where WT-PDS1 (SEQ ID NOS: 8-9) and WT-PDS2 (SEQ ID NOS: 10-11) are two PDS homologous genes of the wild type of pakchoi: A, B, C, D, E in A-PDS1 (SEQ ID NOS: 12-13), A-PDS2 (SEQ ID NOS: 14-15), B-PDS1 (SEQ ID NOS: 16-17), B-PDS2 (SEQ ID NOS: 18-19), C-PDS1 (SEQ ID NOS: 20-21), C-PDS2 (SEQ ID NOS: 22-23), D-PDS1 (SEQ ID NOS: 24-25), D-PDS2 (SEQ ID NOS: 26-27), and E-PDS2 (SEQ ID NOS: 28-29) represent five different mutant strains, respectively, and PDS1 and PDS2 are two PDS homologous genes of the pakchoi: bold fonts represent a target site of the PDS gene; underlines represent substitutions and insertions.

### DETAILED DESCRIPTION OF THE EMBODIMENTS

The present disclosure provides a gene editing vector, including a Cas9 vector backbone and DRs, the DRs being ZmWUS2, AtIPT, and AtPLT5.

In an embodiment of the present disclosure, the gene editing vector further includes a visual marker gene. In an embodiment of the present disclosure, the visual marker gene includes Ruby. In an embodiment of the present disclosure, the Cas9 vector backbone includes pYLCRISPR/Cas9P35S-N.

The present disclosure further provides a gene editing system, where an sgRNA of a target gene is ligated to the gene editing vector. In an embodiment of the present disclosure, the target gene includes a PDS gene. In an embodiment of the present disclosure, sgRNA sequences for the PDS gene includes: PDSsgRNA1: GGAACAACGAGATGCTGACA (SEQ ID NO: 1); and PDSsgRNA2: GCTGCATGGAAGGATGAAGA (SEQ ID NO: 2).

The present disclosure further provides use of the gene editing vector or the gene editing system in genetic transformation, gene editing, and/or tissue culture of a Brassica crop.

The present disclosure further provides a method for genetic transformation and/or gene editing of a Brassica crop, including: ligating an sgRNA designed based on a sequence from a target gene of the Brassica crop with the gene editing vector, transforming a resulting ligation product into Agrobacterium rhizogenes to infect an explant of the Brassica crop, and then subjecting the explant to callus induction and adventitious bud regeneration in sequence to obtain a gene-edited plant.

In an embodiment of the present disclosure, the explant is selected from the group consisting of a petiolate cotyledon and a hypocotyl. In an embodiment of present disclosure, the target gene of the Brassica crop includes a PDS gene. In an embodiment of the present disclosure, sgRNA sequences for the PDS gene includes: PDSsgRNA1: GGAACAACGAGATGCTGACA (SEQ ID NO: 1); and PDSsgRNA2: GCTGCATGGAAGGATGAAGA (SEQ ID NO: 2). In an embodiment of present disclosure, the Brassica crop is selected from the group consisting of Brassica rapa ssp. chinensis, Brassica rapa ssp. pekinensis, Brassica oleracea var. capitata, Brassica oleracea var. italica, and Brassica napus. In an embodiment of the present disclosure, the Agrobacterium rhizogenes includes Agrobacterium rhizogenes K599.

In a previous study, the inventors have observed that Agrobacterium rhizogenes transformation can lead to transgenic roots with fluorescent signals. The callus produced at the wound of the stem segment of the explant can also be observed to have obvious GFP fluorescent signals, but the GFP signals may be affected by some external factors to produce fluorescence. Therefore, the visual marker gene Ruby (the biosynthesis of Ruby only requires three genes CYP76AD1, BvDODA1, and cDOPA5GT) was selected for indication (Yubing He, A non-destructive screening system for plant genetic transformation visible to the naked eye under visible light, and construction method and use thereof, CN patent application No. 202010934705.6). It is extremely difficult to induce regeneration of shoots from transgenic roots of Brassica crops, but as DRs gradually show their great effects, it becomes possible to obtain new transgenic plants of pakchoi (Brassica rapa ssp. chinensis) through Agrobacterium rhizogenes.

In the present disclosure, genetic transformation and a gene editing system for Brassica crop are established by Agrobacterium rhizogenes-mediated delivery of three DRs, ZmWUS2, AtIPT, and AtPLT5. The combination of three DRs, ZmWUS2, AtIPT and AtPLT5, has greatly improved the genetic transformation efficiency of Brassica crops. The Agrobacterium rhizogenes-mediated delivery of three DRs, ZmWUS2, AtIPT, and AtPLT5, can induce explants to form callus and then directly form buds. The method solves the problem of difficult root regeneration for the Brassica crop, and enables species with a poor root regeneration ability to achieve efficient transformation.

Compared with the prior art, the present disclosure has the following advantages:

First of all, a combination of three DRs is used to successfully establish a genetic transformation and gene editing system for Brassica crops mediated by Agrobacterium rhizogenes. This genetic transformation and genome editing system for Brassica crops is highly efficient and independent of genotype, laying the foundation for biological breeding of Brassica crops.

Secondly, the genetic transformation of Brassica crops is currently highly dependent on genotypes, with an extremely poor efficiency of only about 1% to 2%. The entire transformation process may take 6 to 12 months, and the complex hormone ratio and long transformation process require a lot of time and labor costs. However, the system in the present disclosure has a transformation efficiency of 20% to 40%, which is nearly 20 times higher. The transformation time is shortened to 2 to 3 months, saving nearly half the time. In addition, the method is simple to operate and does not require complicated hormone ratios and antibiotic screening, which greatly saves manpower and material resources and provides a basis for gene editing and biological breeding of Brassica crops.

Finally, after obtainment of transgenic roots through Agrobacterium rhizogenes infection, the transgenic roots are subjected to induced bud regeneration to obtain complete plants, which is highly effective for species with strong root regeneration ability. However, Brassica crops have almost no root regeneration ability. The addition of DRs skips the problem of difficult root regeneration, allowing species with poor root regeneration ability to achieve efficient transformation, which is of great reference value for plants with poor root regeneration.

The preferred embodiments of the present disclosure will be described in detail below in conjunction with examples. It should be understood that the following examples are merely for the purpose of illustration, but are not construed as limiting the scope of the present disclosure. Those skilled in the art can make various modifications and substitutions to embodiments without departing from the purpose and spirit of the present disclosure.

All experimental methods used in the following examples are conventional methods, unless otherwise specified.

The materials, reagents, and the like used in the following examples are all commercially available, unless otherwise specified.

### Example 1

1. Construction of Overexpression Vector and Gene Editing Vector and Transformation with Agrobacterium rhizogenes
1.1 Design of sgRNA for Target Gene

In this example, the homologous regions of the PDS gene sequences that could simultaneously edit Chinese cabbage (Brassica rapa), broccoli (Brassica oleracea var. italica), cabbage (Brassica oleracea var. capitata), and rapeseed (Brassica napus) were selected, and the sgRNA was designed using the design tool of Huazhong Agricultural University cbi.hzau.edu.cn/crispr/. The primer sequences are shown in Table 1, and then an expression cassette of the sgRNA was artificially synthesized.

TABLE 1
Sequences of sgRNA

| | |
|---|---|
| PDSsgRNA1 | GGAACAACGAGATGCTGACA |
| | (SEQ ID NO: 1) |
| PDSsgRNA2 | GCTGCATGGAAGGATGAAGA |
| | (SEQ ID NO: 2) |

### 1.2 Construction of CRISPR/Cas9 Gene Editing Vector Containing Three DRs: ZmWUS2, AtIPT, and AtPLT5

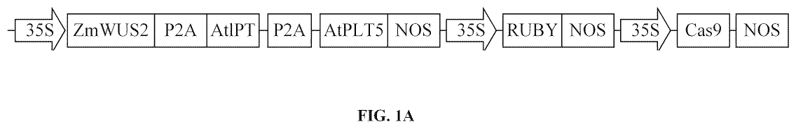

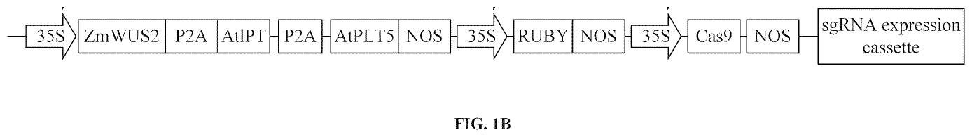

A gene editing vector pYLCRISPR/Cas9P35S-N(Addgene Plasmid #66191) from the research group of Academician Yaoguang Liu of South China Agricultural University was used as backbone. Based on the full-length sequences of ZmWUS2 (GenBank: MP934945.1), AtIPT (AT3G23630), and AtPLT5 (AT5G57390), a 35S-ZmWUS2-P2A-AtIPT-P2A-AtPLT5-NOS expression framework (SEQ ID NO: 7) ligated with the full-length sequences of ZmWUS2, AtIPT, and AtPLT5 was synthesized by GenScript. The expression framework was constructed into a pYLCRISPR/Cas9P35S-N vector using the BamHI restriction site to obtain pYLCRISPR/Cas9-WIP, while a 35S-Ruby-NOS expression framework was PCR amplified from Professor Yubing He's Ruby overexpression vector (Addgene Plasmid #160908) and then ligated to the above vector using the Pmel restriction site for homologous recombination to obtain pYLCRISPR/Cas9-WIP-Ruby. The expression cassette of synthesized sgRNA was inserted into the vector using the AscI restriction site to obtain the PDS gene editing vector, as shown in FIGS. 1A-1B.

A CRISPR/Cas9 gene editing vector plasmid was transformed into Agrobacterium rhizogenes K599 by freeze-thaw method, and positive clones were picked from the plate and shaken at 28° C. and 250 rpm with 1 mL of LB medium (containing 50 mg/L kanamycin and 50 mg/L streptomycin).

### 1.3 Explant Culture

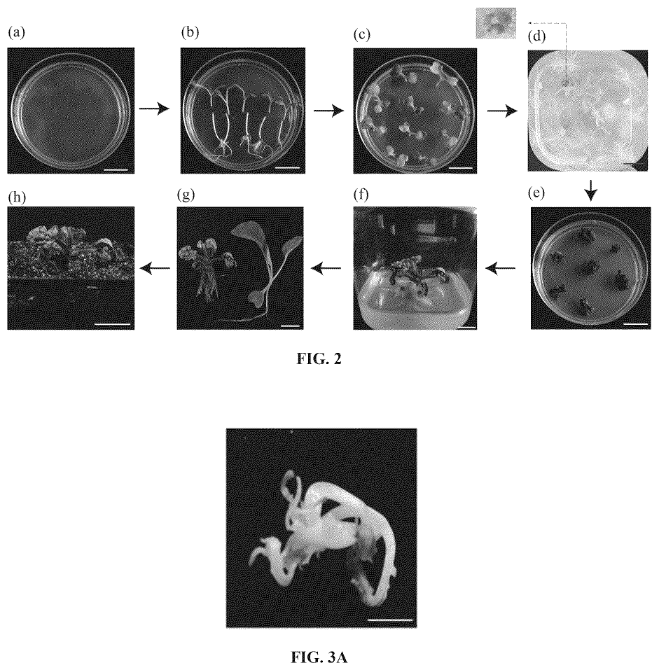

The plump seeds from pakchoi, Chinese cabbage, cabbage, broccoli and rapeseed were selected, disinfected with 75% alcohol for 1 min, treated with 10% sodium hypochlorite for 10 min, and rinsed with sterile water 4 times. The seeds were dried on sterile filter paper and inoculated in 1/2MS medium to allow culture under 12 h of light with 2,000 1× to 3,000 1× of light intensity at 25° C. ((a) in FIG. 2). After 5 d to 6 d, when the cotyledons were fully expanded, the petiolate cotyledons and hypocotyls were cut out as explants ((b) in FIG. 2).

### 1.4 Preparation of Agrobacterium Bacterial Solution

A single colony of the constructed overexpression and gene editing vector was placed in 5 mL of LB liquid medium (containing 50 mg/L kanamycin and 50 mg/L streptomycin), shaken overnight at 28° C. and 200 rpm, and PCR was conducted using the bacterial solution as a template to verify its correctness. The next day, 500 μL of the bacterial solution was transformed into 50 mL of fresh LB liquid medium (containing 50 mg/L kanamycin and 50 mg/L streptomycin) and shaken at 28° C. and 200 rpm overnight. When the bacterial solution had an OD600 value of 0.5 to 0.6, the bacterial solution was centrifuged at 4,000 rpm for 10 min, a resulting supernatant was discarded, and bacterial cells were resuspended and diluted to 50 mL with MS liquid medium (pH=5.2) (Acetosyringone was added to a final concentration of 100 μM), and then shaken at 28° C. and 200 rpm for 4 h for explant infection and transformation.

### 1.5 Genetic Transformation and Regeneration of Plants

The petiolate cotyledons and hypocotyls cultured in step 1.3 were placed in the Agrobacterium bacterial solution prepared in step 1.4 and shaken to allow infection for 5 min, and the excess Agrobacterium bacterial solution was absorbed with sterile filter paper. Another sterile filter paper was laid flat on the MS solid medium, soaked with MS liquid medium, the infected hypocotyl were placed to ensure that the incision of hypocotyl contacted the filter paper, and then cultured in the dark at 25° C. for 24 h to 36 h ((c) in FIG. 2).

After co-cultivation, the hypocotyls were rinsed 4 times with sterile distilled water, dried with sterile filter paper, and transferred to MS solid medium. After 14 d, the explants formed callus tissue ((d) in FIG. 2). After 25 d, adventitious buds differentiated from the explants were transferred to MS solid medium to allow 2 to 3 times of differentiation culture, each time for 20 d ((e) in FIG. 2). A negative control was established by growing untransformed adventitious buds on normal medium. The leaves of the transgenic plants could eventually turn red, while the adventitious buds of the untransformed control group were normal green. The number of induced calli, the number of red calli, and the number of red adventitious buds of the pakchoi, Chinese cabbage, cabbage, broccoli, rapeseed were counted respectively, and the genetic transformation efficiency was calculated according to: number of red adventitious buds/number of inoculated explants×100%. The results showed that the conversion rate was 21% to 40%, with broccoli having the highest rate. The red positive seedlings were transferred to MS solid medium for propagation and finally cultured for rooting until complete regenerated plants were produced ((f) and (g) in FIG. 2). The plants with desirable growth were selected to allow domestication in opened bottles, the medium on the roots was removed by washing, and the seedlings were transferred into nutrient pots filled with culture soil (peat soil: vermiculite=3:1) ((h) in FIG. 2), the pots were covered with plastic film to keep moisture for 2 d to 3 d, and then the seedlings were managed normally. Transgenic seeds were obtained after the T₀ generation plants were overwintered and vernalized.

TABLE 2
Genetic transformation efficiency of different Brassica crops

| | Number of | Number of | Number | Number | |
|---|---|---|---|---|---|
| | inoculated | induced | of red | of red | |
| | explants | calli | calli | adventitious | Conversion |
| Genotype | (explant) | (callus) | (callus) | buds | efficiency |
| Pakchoi | 100 | 20 | 7 | 21 | 21% |
| Chinese | 100 | 22 | 9 | 23 | 23% |
| cabbage | | | | | |
| Cabbage | 100 | 26 | 10 | 36 | 36% |
| Broccoli | 100 | 28 | 10 | 40 | 40% |
| Rapeseed | 100 | 23 | 9 | 31 | 31% |

### 2. Identification of Gene-Edited Mutants

Primers were designed based on the mutation regions of the 2 PDS genes Bra032770 and Bra010751 of pakchoi (Table 3). The DNA of the induced transgenic plants from pakchoi was extracted and amplified by PCR using Taq enzyme and sent to a biological company for sequencing. The sequencing results were analyzed using DNAMAN software to determine the mutation type and specific location of the mutation, thereby inferring the specific mutation type of each individual plant.

TABLE 3
Primer sequences for gene editing site
detection

| | |
|---|---|
| Bra032770F | AGAACAAGGCGAAGGACTGCTG |
| | (SEQ ID NO: 3) |
| Bra032770R | TGCATTGACAGTTCGTTCGGGT |
| | (SEQ ID NO: 4) |
| Bra010751F | AACAAGGAGGAGGAGGAGTGCT |
| | (SEQ ID NO: 5) |
| Bra010751R | TGTCACCCGATCAGGTACTCCC |
| | (SEQ ID NO: 6) |

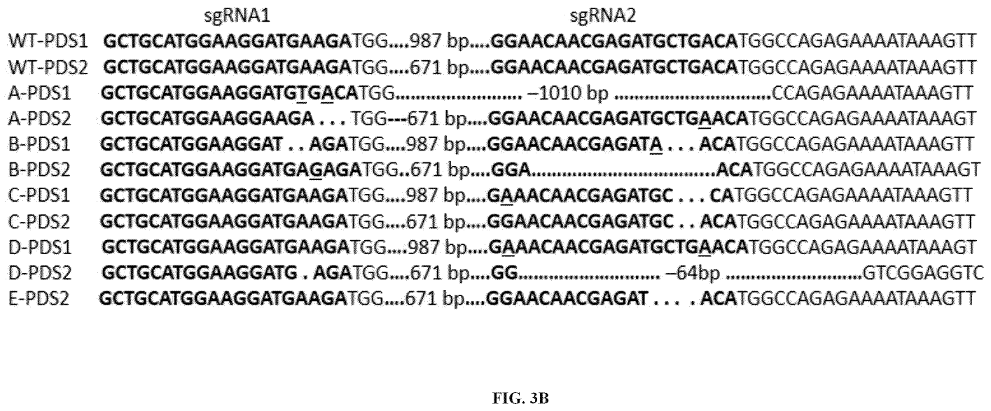

The results showed that 7 PDS gene-edited albino plants were finally obtained from 11 red transgenic buds, showing a gene editing efficiency of 63.63%. By TA cloning and sequencing on the PCR products of 5 albino plants and analyzing the mutation types and frequencies, 4 homozygous mutations and 1 heterozygous mutation were detected. The mutations were mainly insertions or deletions, among which the PDS sequence of mutation A had a significant deletion of 1,010 bp. Among the two target sites, the mutation frequency of site 2 was higher at 90%, while the mutation frequency of site 1 was 50% (FIGS. 3A-3B).

1. A gene editing vector, comprising a clustered regularly interspaced short palindromic repeats-associated protein 9 (Cas9) vector backbone and further comprising developmental regulators (DRs), the DRs being ZmWUS2, AtIPT, and AtPLT5.

2. The gene editing vector according to claim 1, further comprising a visual marker gene.

3. The gene editing vector according to claim 2, wherein the visual marker gene comprises Ruby.

4. The gene editing vector according to claim 1, wherein the Cas9 vector backbone comprises pYLCRISPR/Cas9P35S-N.

5. A gene editing system, wherein a single guide RNA (sgRNA) of a target gene is ligated to the gene editing vector according to claim 1.

6. The gene editing system according to claim 5, wherein the target gene comprises a PDS gene.

7. The gene editing system according to claim 6, wherein sgRNA sequences for the PDS gene comprises:
PDSsgRNA1:
(SEQ ID NO: 1)
GGAACAACGAGATGCTGACA;
and
PDSsgRNA2:
(SEQ ID NO: 2)
GCTGCATGGAAGGATGAAGA.

8. (canceled)

9. A method for culturing a Brassica crop tissue, comprising contacting the gene editing vector according to claim 1 with the Brassica crop tissue.

10. A method for genetic transformation and/or gene editing of a Brassica crop, comprising: ligating an sgRNA designed based on a sequence from a target gene of the Brassica crop with the gene editing vector according to claim 1, transforming a resulting ligation product into Agrobacterium rhizogenes to infect an explant of the Brassica crop, and then subjecting the explant to callus induction and adventitious bud regeneration in sequence to obtain a gene-edited plant.

11. The method according to claim 10, wherein the explant is selected from the group consisting of a petiolate cotyledon and a hypocotyl.

12. The method according to claim 10, wherein the target gene of the Brassica crop comprises a PDS gene.

13. The method according to claim 12, wherein sgRNA sequences for the PDS gene comprises:
PDSsgRNA1:
(SEQ ID NO: 1)
GGAACAACGAGATGCTGACA;
and
PDSsgRNA2:
(SEQ ID NO: 2)
GCTGCATGGAAGGATGAAGA.

14. The method according to claim 10, wherein the Brassica crop is selected from the group consisting of Brassica rapa ssp. chinensis, Brassica rapa ssp. pekinensis, Brassica oleracea var. capitata, Brassica oleracea var. italica, and Brassica napus.

15. The method according to claim 10, wherein the Agrobacterium rhizogenes comprises Agrobacterium rhizogenes K599.

16. The method according to claim 9, wherein the gene vector further comprises a visual marker gene.

17. The method according to claim 16, wherein the visual marker gene comprises Ruby.

18. The method according to claim 9, wherein the Cas9 vector backbone comprises pYLCRISPR/Cas9P35S-N.
